(19) United States
(12) Patent Application Publication
(10) Pub. No.: US 2024/0425459 A1
(43) Pub. Date: Dec. 26, 2024

(54) METHOD FOR SYNTHESIZING AN OXIME COMPRISING AN IMIDAZOLE GROUP AND METHOD FOR SYNTHESIZING A NITRILE OXIDE FROM SAID OXIME

(71) Applicant: COMPAGNIE GENERALE DES ETABLISSEMENTS MICHELIN, Clermont-Ferrand (FR)

(72) Inventors: FRANCOIS JEAN-BAPTISTE-DIT-DOMINIQUE, Clermont-Ferrand (FR); SERGEY IVANOV, Clermont-Ferrand (FR); OLEG UGOLNIKOV, Clermont-Ferrand (FR); STEPHANE LE ROUX, Ecouche Les Vallees (FR); JEAN CHRISTOPHE JOSEPH, Limay (FR); PATRICE ROOL, Riom (FR); Julien Michaux, Riom (FR)

(21) Appl. No.: 18/722,304

(22) PCT Filed: Dec. 19, 2022
(86) PCT No.: PCT/EP2022/086547

§ 371 Date: Jun. 20, 2024

(30) Foreign Application Priority Data
Dec. 21, 2021 (FR) 2114104

(51) Int. Cl. C07D 233/61

(52) U.S. Cl. CPC C07D 233/61

(57) ABSTRACT

A novel process for synthesizing a compound of formula (I)

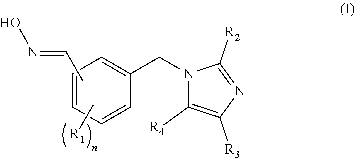

comprises: a step A1 of obtaining an intermediate compound of formula (V) via a halomethylation reaction of an aromatic aldehyde of formula (VI) in the presence of a halomethylation agent; a step A2 of obtaining an intermediate compound of formula (III) via nucleophilic substitution of the intermediate compound of formula (V) with an imidazole compound of formula (IV); and a step A3 of obtaining the compound of formula (I) via a condensation reaction of the intermediate compound of formula (III) with hydroxylamine (II), these steps being carried out without isolation of the intermediate compound of formula (III), the intermediate compound of formula (V), or both. A nitrile oxide is also synthesized from the compound of formula (I).

The present invention relates to a process for synthesizing an oxime bearing an imidazole group (hereinafter referred to as compound of formula (I)) and to a process for synthesizing a nitrile oxide bearing an imidazole group (hereinafter referred to as compound of formula (X)) via the synthesis of the compound of formula (I) according to the synthesis process of the invention.

Nitrile oxides comprising an imidazole group are used as agents for modifying diene elastomers. They make it possible, once grafted to the carbon-carbon double bonds of these diene elastomers, to modify their structure and ultimately to improve the mechanical properties of elastomeric compositions comprising such grafted diene elastomers and at least one reinforcing filler. These nitrile oxides are described in the document WO2015059269.

At present, in the synthesis of these nitrile oxides, it is necessary to carry out long and numerous steps of separating, purifying, isolating and drying each of the synthesis intermediates in order to avoid the presence of impurities which lead to secondary reactions that have a negative impact on the selectivity of the reaction and on its yield.

Furthermore, the prior art process for synthesizing these nitrile oxides uses raw materials and/or solvents that are very expensive to purchase. For example, the second step of the prior art process described in the examples of WO2015059269 employs a step of formylation in the presence of titanium tetrachloride and dichloromethyl methyl ether; the third step is carried out in the presence of N,N-dimethylformamide. The latter compound is used as solvent and in a large amount. Moreover, its use is recommended less and less by national regulations. The use of these solvents imposes additional constraints in terms of safety, handling and treatment of discharges for the environment, making this synthesis process complex and expensive, in particular for a synthesis on an industrial scale.

Consequently, there is a need to provide an improved process for synthesizing a nitrile oxide compound comprising an imidazole group. More particularly, there is a need for a process for synthesizing a nitrile oxide compound comprising an imidazole group that can be applied on an industrial scale and that makes it possible to reduce the number of steps of isolating synthesis intermediates, to reduce the cycle times, to reduce the amount of solvents used, to reduce the number of different solvents used, to increase the yields by limiting the formation of by-products and ultimately to reduce the production cost of these nitrile oxides.

The aim of the present invention is to meet this need and to provide a synthesis process that is improved compared to that of the prior art.

Continuing its research, the applicant has developed a novel process for synthesizing an oxime bearing an imidazole group as well as a process for synthesizing a nitrile oxide from said oxime synthesized according to the process of the invention; this process having a synthesis yield that is significantly improved compared to the process of the prior art and also making it possible to reduce the number of solvents and the amount thereof.

Thus, the present invention relates to a process for synthesizing a compound of formula (I)

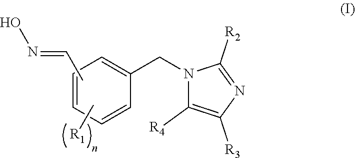

in which:
R₁ represents, independently of one another, a C1-C6 alkyl, preferably a C1-C3 alkyl;
R₂ represents a hydrogen atom, a C1-C12 alkyl, preferably a C1-C6 alkyl, more preferentially a C1-C3 alkyl;
R₃ and R₄, independently of one another, represent a hydrogen atom, a C1-C12 alkyl, (preferably a C1-C6 alkyl, more preferentially a C1-C3 alkyl) or form, together with the carbon atoms to which they are attached, a ring, in particular an aromatic ring, preferably phenyl; and
n is an integer equal to 0, 1, 2, 3 or 4;
said synthesis process comprising the following successive steps A1, A2, A3:
   a step A1 of obtaining an intermediate compound of formula (V) via a halomethylation reaction of an aromatic aldehyde of formula (VI) in the presence of a halomethylation agent according to the following reaction scheme:

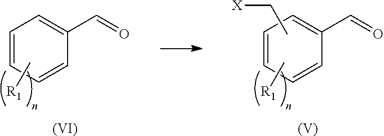

with R₁ and n as defined above; and X being a halogen atom, preferably a bromine or chlorine atom, more preferentially a chlorine atom;
a step A2 of obtaining an intermediate compound of formula (III) via nucleophilic substitution of the preceding intermediate compound of formula (V) with an imidazole compound of formula (IV) according to the following reaction scheme:

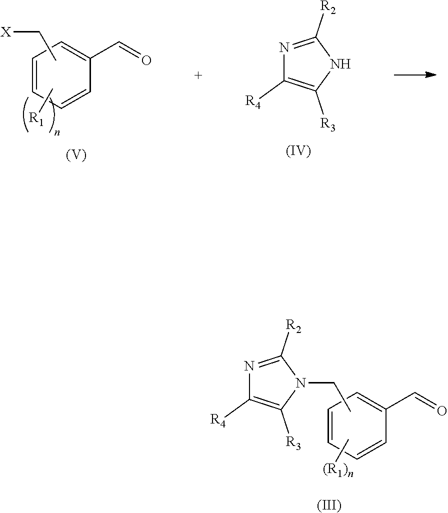

with n, X, R₁, R₂, R₃ and R₄ as defined above;
a step A3 of obtaining the compound of formula (I) via a condensation reaction of the preceding intermediate compound of formula (III) with hydroxylamine (11) according to the following reaction scheme:

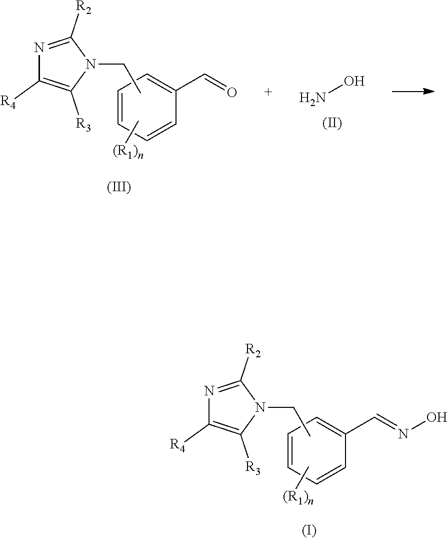

with R₁, R₂, R₃, R₄ and n as defined above; and
steps A1, A2, A3 being carried out without isolation of at least one intermediate compound chosen from the group formed by the intermediate compound of formula (III) and the intermediate compound of formula (V).

Preferentially, the halomethylation agent is preheated to a temperature T1 within a range extending from 23° C. to 50° C., more preferentially within a range extending from 30° C. to 45° C., before bringing it into contact with the compound of formula (VI).

Preferentially, the compound of formula (VI) is added all at once after the preheating of the halomethylation agent.

Preferentially, the halomethylation agent is a mixture of a formaldehyde donor and hydrochloric acid.

Preferentially, the amount of formaldehyde donor is within a range extending from 1 molar equivalent to 7 molar equivalents, preferably from 1.2 molar equivalents to 6 molar equivalents, more preferentially still from 2 molar equivalents to 6 molar equivalents, relative to the amount of compound of formula (VI).

Preferentially, step A1 is carried out at a temperature T2 within a range extending from 60° C. to 100° C., more preferentially within a range extending from 70° C. to 90° C.

Preferentially, an organic solvent S1 is added at the end of step A1 and the organic phase is recovered, the organic solvent S1 being chosen from halogenated solvents.

Preferentially, step A2 is carried out in the presence of an organic solvent S2 chosen from the group consisting of halogenated solvents, dimethyl sulfoxide, acetone, alcohols such as isopropanol, ethers such as tetrahydrofuran, ethyl acetate; preferentially the organic solvent S2 is a halogenated organic solvent, more preferentially a chlorinated organic solvent.

Preferentially, the organic solvent S2 is identical to the organic solvent S1.

Preferentially, the amount of compound of formula (IV) is within a range extending from 1.1 molar equivalents to 6 molar equivalents, more preferentially extending from 2 molar equivalents to 4 molar equivalents, relative to the compound of formula (V).

Preferentially, step A2 is carried out at a temperature which is less than or equal to the reflux temperature of the organic solvent S2.

Preferentially, the amount of organic solvent S2 is at least 0.5 volume relative to 1 volume of organic solvent S1, more preferentially is within a range extending from 1 volume to 10 volumes relative to one volume of organic solvent S1.

Preferentially, step A3 is carried out in an organic solvent S3 chosen from the group consisting of halogenated solvents, acetone, alcohols such as isopropanol, ethers such as tetrahydrofuran, ethyl acetate; preferentially the organic solvent S3 a halogenated organic solvent, preferably is a chlorinated organic solvent.

Preferentially, the organic solvent S3 is identical to the organic solvent S2.

The compounds of formula (I) obtained according to the synthesis process of the invention can be converted into corresponding nitrile oxides while retaining a good production yield and in particular a yield that is greater than the yield of the prior art processes for synthesizing nitrile oxides.

The invention also relates to a process for synthesizing a compound of formula (X), said process comprising the following steps:
a step (i) of synthesizing the compound of formula (I) according to process described above,
a step (ii) of converting the compound of formula (I) into a compound of formula (X) in the presence of at least one organic solvent S4 and at least one oxidant according to the following reaction scheme:

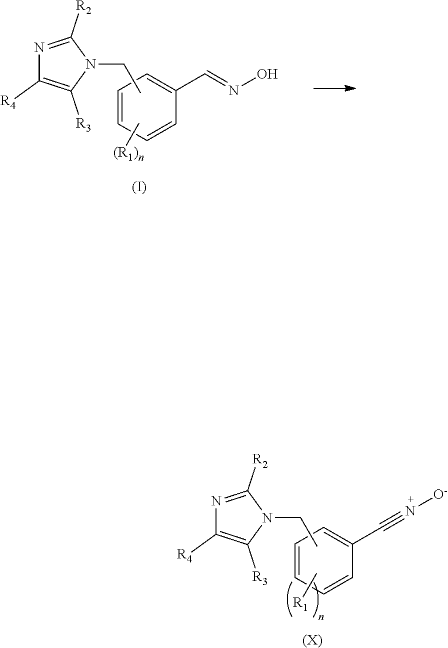

where:
   R₁ represents, independently of one another, a C1-C6 alkyl, preferably a C1-C3 alkyl;
   R₂ represents a hydrogen atom, a C1-C12 alkyl, preferably a C1-C6 alkyl, more preferentially a C1-C3 alkyl;
   R₃ and R₄, independently of one another, represent a hydrogen atom, a C1-C12 alkyl, (preferably a C1-C6 alkyl, more preferentially a C1-C3 alkyl) or form, together with the carbon atoms to which they are attached, a ring, preferably an aromatic ring, preferably phenyl; and
   n is an integer equal to 0, 1, 2, 3 or 4; and
a step (iii) of recovering the compound of formula (X).

The synthesis process according to the invention thus makes it possible to reduce the number of chemical steps compared to the prior art synthesis process, while using more acceptable solvents, with optimized synthesis yields. The use in particular of an aromatic aldehyde as starting raw material and in particular of suitable solvents makes it possible to link steps A1, A2 and A3 without the need to isolate at least one synthesis intermediate obtained during steps A1 and A2. Thus, halomethylation of the compound of formula (VI) is carried out, and then followed directly by a nucleophilic substitution with a compound of formula (IV), which itself is followed directly by a condensation reaction with hydroxylamine thus resulting in the oxime of formula (I) having a satisfactory level of purity and yield, and doing so without isolation of the intermediate compounds of formula (V) and/or of formula (111), preferably without isolation of the intermediate compounds of formula (V) and of formula (111). The synthesis process according to the invention advantageously makes it possible to avoid the drying steps and to limit the steps of separating and purifying these synthesis intermediate compounds. Finally, the overall cycle time is improved since no drying of the synthesis intermediates is carried out. The synthesis process according to the invention is therefore more economical, faster and more environmentally friendly.

The process according to the invention can be applied on an industrial scale and makes it possible in particular to obtain cumulative yields of at least 60% for the sequence without isolation of the intermediates of steps A1 and A2.

Furthermore, the synthesis process according to the invention also has the advantage of reducing the number and amount of solvents and also of using common solvents such as alcoholic solvents that are more environmentally friendly and safer for operators than for example N,N-dimethylformamide.

In the present application, unless expressly indicated otherwise, all the percentages (%) shown are percentages (%) by mass.

Moreover, any interval of values denoted by the expression “between a and b” represents the range of values extending from more than a to less than b (that is to say, limits a and b excluded), whereas any interval of values denoted by the expression “from a to b” means the range of values extending from a up to b (that is to say, including the strict limits a and b).

The compounds mentioned in the description may be of fossil origin or be biobased. In the latter case, they may be partially or completely derived from biomass or may be obtained from renewable raw materials derived from biomass. Obviously, the compounds mentioned may also originate from the recycling of already-used materials, that is to say that they may be partially or completely derived from a recycling process, or else obtained from starting materials themselves derived from a recycling process. This notably relates to polymers, plasticizers, fillers, etc.

“C1-Cx alkyl” is understood to mean, within the meaning of the present invention, a saturated and linear or branched hydrocarbon chain comprising 1 to x carbon atoms. Mention may be made, by way of example, of the methyl, ethyl, propyl, isopropyl, butyl, isobutyl, sec-butyl, tert-butyl, pentyl or hexyl groups.

“Aromatic ring” is understood to mean, within the meaning of the present invention, an aromatic hydrocarbon group comprising from 6 to 20 carbon atoms, and comprising one or more fused rings, such as a phenyl or naphthyl group. Advantageously, it is phenyl (C6 aryl).

“Ambient temperature” is understood to mean, within the meaning of the present invention, a temperature within a range extending from 20° C. to 25° C., more preferentially still a temperature of 23° C.

The expressions “intermediate compound”, “synthesis intermediate”, “synthesis intermediate compound” are interchangeable and denote a chemical compound intended to be chemically converted to create another compound. In that sense, the intermediate should no longer be present in the final product, or at least only as a residual impurity.

“Halogen atom” is understood to mean, within the meaning of the present invention, an atom chosen from the group formed by chlorine, bromine, iodine and fluorine. More preferentially, it is a bromine or chlorine atom, more preferentially still a chlorine atom.

A first subject of the present invention relates to a process for synthesizing a compound of formula (I)

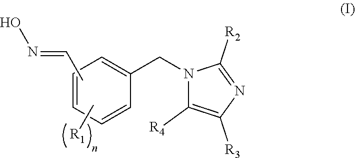

in which:
R₁ represents, independently of one another, a C1-C6 alkyl, preferably a C1-C3 alkyl;
R₂ represents a hydrogen atom, a C1-C12 alkyl, preferably a C1-C6 alkyl, more preferentially a C1-C3 alkyl;
R₃ and R₄, independently of one another, represent a hydrogen atom, a C1-C12 alkyl, (preferably a C1-C6 alkyl, more preferentially a C1-C3 alkyl) or form, together with the carbon atoms to which they are attached, a ring, in particular an aromatic ring, preferably phenyl; and
n is an integer equal to 0, 1, 2, 3 or 4; and said synthesis process comprising the following successive steps A1, A2, A3:
   a step A1 of obtaining an intermediate compound of formula (V) via a halomethylation reaction of an aromatic aldehyde of formula (VI) in the presence of a halomethylation agent according to the following reaction scheme:

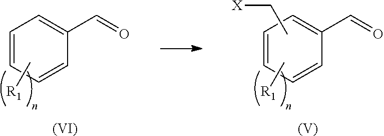

with R₁ and n as defined above; and X being a halogen atom, preferably a bromine or chlorine atom, more preferentially a chlorine atom;
a step A2 of obtaining an intermediate compound of formula (III) via nucleophilic substitution of the preceding intermediate compound of formula (V) with an imidazole compound of formula (IV) according to the following reaction scheme:

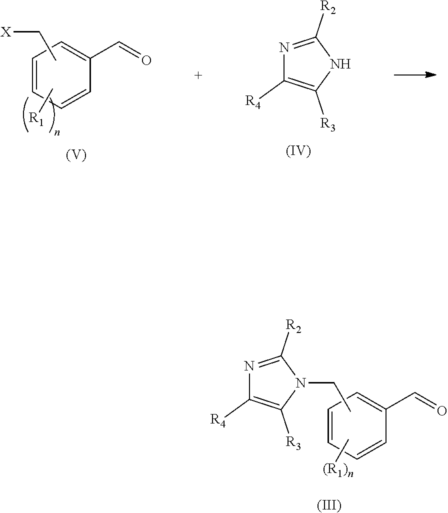

with n, X, R₁, R₂, R₃ and R₄ as defined above;
a step A3 of obtaining the compound of formula (I) via a condensation reaction of the preceding intermediate compound of formula (III) with hydroxylamine (11) according to the following reaction scheme:

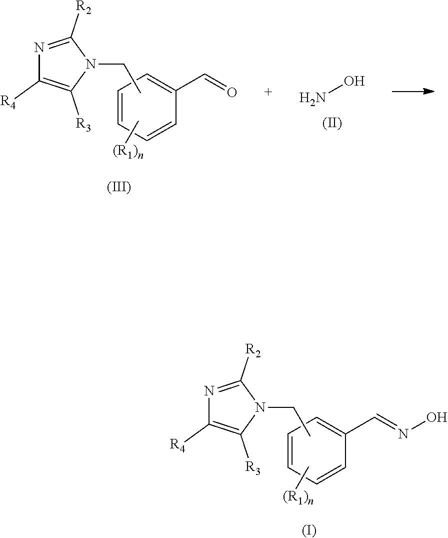

with R₁, R₂, R₃, R₄ and n as defined above; and
steps A1, A2, A3 being carried out without isolation of at least one intermediate compound chosen from the group formed by the intermediate compound of formula (III) and the intermediate compound of formula (V).

Preferentially, the process of the invention comprises a sequence of reactions A1, A2 and A3, in which the synthesis intermediate compound (V) resulting from the halomethylation reaction (step A1) is neither isolated nor dried before proceeding to the nucleophilic substitution reaction with an imidazole compound of formula (IV) (step A2). Preferentially, the synthesis intermediate compound of formula (III) resulting from the nucleophilic substitution reaction with an imidazole compound (step A2) is neither isolated nor dried before proceeding to the condensation reaction with hydroxylamine (step A3). More preferentially still, neither the synthesis intermediate compound of formula (V) nor the synthesis intermediate compound of formula (III) are isolated or dried during the sequence of steps A1 to A2 and A2 to A3.

The expression “without isolation” signifies, within the meaning of the present invention, that in a synthesis process or a reaction sequence, a synthesis intermediate undergoes several successive and/or simultaneous reactions in its reaction medium, by limiting the separation steps and by eliminating the steps of purifying and drying the synthesis intermediate compounds.

The expression “reaction medium” denotes a medium in which the chemical reactions take place.

The term “isolation” denotes the separation of a synthesis intermediate compound from the reaction medium, optionally followed by its purification and/or its drying. The methods for separating and/or purifying an intermediate compound are well known to those skilled in the art. Mention may for example be made of filtration, chromatography, centrifugation, solvent extraction, distillation, etc. Washing with water will not be considered as a separation method nor as a purification method within the meaning of the present invention and therefore does not lead to isolation of a synthesis intermediate.

In the synthesis process of the invention, the first step, step A1, makes it possible to obtain an intermediate compound of formula (V) via a halomethylation reaction of an aromatic aldehyde of formula (VI) in the presence of a halomethylation agent according to the following reaction scheme:

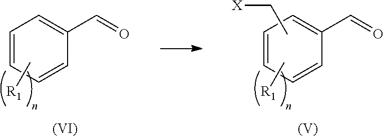

in which:
R₁ represents, independently of one another, a C1-C6 alkyl, preferably a C1-C3 alkyl;
n is an integer equal to 0, 1, 2, 3 or 4; and
X is a halogen atom chosen from the group consisting of chlorine, bromine, fluorine, iodine; more preferentially chosen from a chlorine atom and a bromine atom; more preferentially still is a chlorine atom.

More preferentially, in the compounds of formula (VI) and (V), n is an integer equal to 3; R₁ represents, independently of one another, a C1-C6 alkyl, preferably a C1-C3 alkyl; and X is a halogen atom chosen from the group consisting of chlorine, bromine, fluorine and iodine, more preferentially chosen from a chlorine atom and a bromine atom, more preferentially still is a chlorine atom. More preferentially still, n is an integer equal to 3; R₁ is a C1-C3 alkyl; and X is chosen from a chlorine atom or a bromine atom, preferably a chlorine atom. Even more preferably, n is equal to 3; the R₁ groups are identical, located in the ortho and para position with respect to the aldehyde function and are a C1-C3 alkyl, preferably methyl, ethyl or propyl; and X is chosen from a chlorine atom or a bromine atom, preferably a chlorine atom.

Step A1 is a halomethylation reaction. “Halomethylation reaction” is understood to mean a reaction of electrophilic substitution type, followed by a nucleophilic substitution during which an aromatic compound is alkylated with a halomethylation agent in the presence or absence of a catalyst. When the halomethylation agent comprises at least one chlorine atom, reference will then be made to chloromethylation. When the halomethylation agent comprises at least one bromine atom, reference will then be made to bromomethylation.

The halomethylation agent may be any known agent. Preferentially, the halomethylation agent is a formaldehyde donor in a solution of hydrochloric acid or of hydrobromic acid in the presence or absence of a Lewis acid such as zinc halides or of a carboxylic acid such as acetic acid. More preferentially still, the halomethylation agent is a mixture of formaldehyde donor and hydrochloric acid.

Preferentially, in the synthesis process of the invention, the halomethylation agent is preheated to a temperature T1 within a range extending from 23° C. to 50° C., more preferentially within a range extending from 30° C. to 45° C., before bringing it into contact with the compound of formula (VI). The halomethylation agent is brought into contact with the compound of formula (VI) preferentially directly after its preheating.

When the halomethylation agent is a formaldehyde donor, the amount of the formaldehyde donor in step A1 may be within a range extending from 1 molar equivalent to 7 molar equivalents, preferably from 1.2 molar equivalents to 6 molar equivalents, more preferentially still from 2 molar equivalents to 6 molar equivalents, relative to the amount of compound of formula (VI).

Preferentially, the formaldehyde donor may be chosen from paraformaldehyde and formaldehyde. Preferably, the formaldehyde donor is paraformaldehyde.

Preferentially, the solution of hydrochloric acid used in the context of the present invention may be a solution of hydrochloric acid at a concentration within a range extending from 15% to 50% by volume, more preferentially from 27% to 42% by volume, more preferentially from 30% to 40% by volume.

Preferentially, in the synthesis process of the invention, the compound of formula (VI) may be added all at once, in particular directly, after the preheating of the halomethylation agent.

Preferentially, step A1 may be carried out at a temperature T2 within a range extending from 60° C. to 100° C., more preferentially within a range extending from 70° C. to 90° C.

According to one embodiment, an organic solvent S1 may be added at the end of step A1; a step of recovering the organic phase may then be carried out; the organic solvent S1 being able to be chosen from halogenated solvents.

“Halogenated solvent” or “halogenated organic solvent” is understood to mean a solvent comprising at least one halogenated hydrocarbon, that is to say a hydrocarbon comprising one or more halogen substituents. Preferentially, the halogenated solvent is a chlorinated solvent, that is to say a solvent comprising at least one chlorinated hydrocarbon, that is to say a hydrocarbon comprising one or more chlorine substituents. As chlorinated solvent, the organic solvent S1 may be chosen from the group consisting of dichloromethane, dichloroethane, chloroform; preferentially the organic solvent S1 may be dichloromethane.

Preferentially, the intermediate compound of formula (V) is neither isolated nor dried at the end of step A1.

The intermediate compound of formula (V) undergoes a nucleophilic substitution reaction with an imidazole compound of formula (IV) (this is step A2) directly in the same reaction medium as that of step A1.

Thus, the process of the invention comprises a step A2 of obtaining an intermediate compound of formula (III) via nucleophilic substitution of the preceding intermediate compound of formula (V) with an imidazole compound of formula (IV) according to the following reaction scheme:

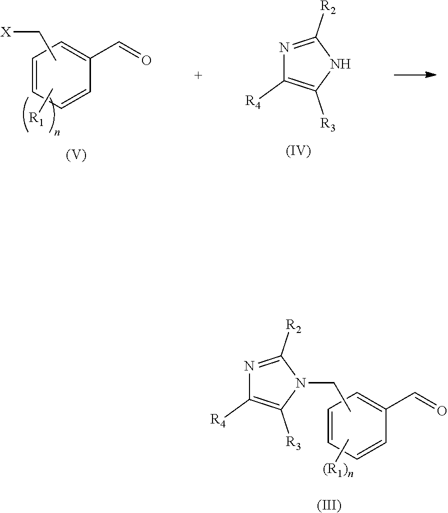

in which:
R₁ represents, independently of one another, a C1-C6 alkyl, preferably a C1-C3 alkyl;
n is an integer equal to 0, 1, 2, 3 or 4;
X is a halogen atom chosen from the group consisting of chlorine, bromine, fluorine, iodine, more preferentially chosen from a chlorine atom and a bromine atom, more preferentially still is a chlorine atom;
R₂ represents a hydrogen atom, a C1-C12 alkyl, preferably a C1-C6 alkyl, more preferentially a C1-C3 alkyl; and
R₃ and R₄, independently of one another, represent a hydrogen atom, a C1-C12 alkyl (preferably a C1-C6 alkyl, more preferentially a C1-C3 alkyl), or form, together with the carbon atoms to which they are attached, a ring, preferably an aromatic ring, more preferentially phenyl.

Preferentially, in the compound of formula (V), n is an integer equal to 3; R₁ represents, independently of one another, a C1-C6 alkyl, preferably a C1-C3 alkyl; and X is a halogen atom chosen from the group consisting of chlorine, bromine, fluorine and iodine, more preferentially chosen from a chlorine atom and a bromine atom, more preferentially still is a chlorine atom. More preferentially still, n is an integer equal to 3; R₁ is a C1-C3 alkyl; and X is chosen from a chlorine atom or a bromine atom, preferably a chlorine atom. Even more preferably, n is equal to 3; the R₁ groups are identical, located in the ortho and para position with respect to the aldehyde function and are a C1-C3 alkyl, preferably methyl, ethyl or propyl; and X is chosen from a chlorine atom or a bromine atom, preferably a chlorine atom.

Preferentially, in the compound of formula (IV), R₂ represents a hydrogen atom or a C1-C6 alkyl, more preferentially a C1-C3 alkyl; and R₃ and R₄ represent a hydrogen atom or a C1-C6 alkyl, more preferentially a C1-C3 alkyl, or form, together with the carbon atoms to which they are attached, a phenyl. More preferentially still, in the compound of formula (IV), R₂ represents a C1-C3 alkyl, preferably methyl, ethyl or propyl; and R₃ and R₄ represent a hydrogen atom or a C1-C3 alkyl (preferably methyl, ethyl, propyl) or form, together with the carbon atoms to which they are attached, a phenyl. More preferentially still, R₂ represents a C1-C3 alkyl, preferably methyl, ethyl or propyl; and R₃ and R₄ are identical and are a hydrogen atom.

Preferentially, in the compound of formula (III), n is an integer equal to 3; R₁ represents, independently of one another, a C1-C6 alkyl, preferably a C1-C3 alkyl; R₂ represents a hydrogen atom or a C1-C6 alkyl, more preferentially a C1-C3 alkyl; and R₃ and R₄ represent a hydrogen atom or a C1-C6 alkyl, more preferentially a C1-C3 alkyl, or form, together with the carbon atoms to which they are attached, a phenyl. More preferentially still, n is an integer equal to 3; R₁ is a C1-C3 alkyl; R₂ represents a C1-C3 alkyl, preferably methyl, ethyl or propyl; and R₃ and R₄ represent a hydrogen atom or a C1-C3 alkyl (preferably methyl, ethyl or propyl) or form, together with the carbon atoms to which they are attached, a phenyl. Even more preferably, n is equal to 3; the R₁ groups are identical, located in the ortho and para position with respect to the aldehyde function and are a C1-C3 alkyl, preferably methyl, ethyl or propyl; R₂ represents a C1-C3 alkyl, preferably methyl, ethyl or propyl; and R₃ and R₄ are identical and are a hydrogen atom.

Step A2 may be carried out in the presence of an organic solvent S2 chosen from the group consisting of halogenated solvents, dimethyl sulfoxide, acetone, alcohols such as isopropanol, ethers such as tetrahydrofuran, ethyl acetate. Preferentially, the organic solvent S2 is a halogenated solvent, preferably a chlorinated solvent, more preferentially S2 is dichloromethane.

Preferentially, the organic solvent S2 is identical to the organic solvent S1. Thus, when the organic solvent S1 is a halogenated solvent, preferably a chlorinated solvent, more preferentially dichloromethane, then S2 is also a halogenated solvent, preferably a chlorinated solvent, more preferentially dichloromethane.

Preferentially, in step A2, the amount of compound of formula (IV) may be within a range extending from 1.1 molar equivalents to 6 molar equivalents, more preferentially extending from 2 molar equivalents to 4 molar equivalents, relative to the compound of formula (V).

Preferentially, step A2 is carried out at a temperature less than or equal to the reflux temperature of the organic solvent S2.

Preferentially, the amount of organic solvent S2 is at least 0.5 volume relative to 1 volume of organic solvent S1, more preferentially is within a range extending from 1 volume to 10 volumes relative to 1 volume of organic solvent S1.

The process of the invention may optionally comprise, at the end of step A2 and before step A3, a least one step of washing with water. At the end of this washing step, a step of recovering the organic phase can be carried out and the process can continue directly with the performance of step A3.

Preferentially, the intermediate compound of formula (III) is not isolated, and does not undergo any additional purification steps other than optional washings with water at the end of step A2.

The intermediate compound of formula (III) undergoes a condensation reaction with hydroxylamine (this is step A3) directly, in particular, in the same reaction medium as that of step A2.

The synthesis process of the invention comprises a step A3 of obtaining the compound of formula (I) via a condensation reaction of the preceding intermediate compound of formula (III) with hydroxylamine (11) according to the following reaction scheme:

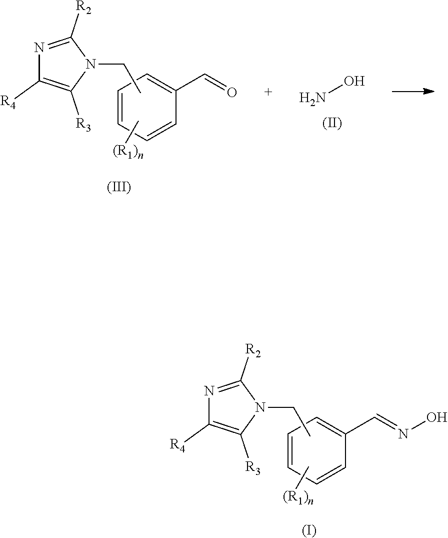

with R₁, R₂, R₃, R₄ and n as defined above, including with their preferred embodiments,

Preferentially, in the compound of formula (III) and in the compound of formula (I), n is an integer equal to 3; R₁ represents, independently of one another, a C1-C6 alkyl, preferably a C1-C3 alkyl; R₂ represents a hydrogen atom or a C1-C6 alkyl, more preferentially a C1-C3 alkyl; and R₃ and R₄ represent a hydrogen atom or a C1-C6 alkyl, more preferentially a C1-C3 alkyl, or form, together with the carbon atoms to which they are attached, a phenyl. More preferentially still, n is an integer equal to 3; R₁ is a C1-C3 alkyl; R₂ represents a C1-C3 alkyl, preferably methyl, ethyl or propyl; and R₃ and R₄ represent a hydrogen atom or a C1-C3 alkyl (preferably methyl, ethyl or propyl) or form, together with the carbon atoms to which they are attached, a phenyl. Even more preferably, n is equal to 3; the R₁ groups are identical, located in the ortho and para position with respect to the aldehyde function, respectively the oxime function, and are a C1-C3 alkyl, preferably methyl, ethyl or propyl; R₂ represents a C1-C3 alkyl, preferably methyl, ethyl or propyl; and R₃ and R₄ are identical and are a hydrogen atom.

Preferentially, step A3 is carried out in an organic solvent S3 chosen from the group consisting of halogenated solvents, acetone, alcohols such as isopropanol, ethers such as tetrahydrofuran, ethyl acetate. Preferentially, the organic solvent S3 is a halogenated organic solvent, preferably a chlorinated organic solvent, more preferentially the organic solvent S3 is dichloromethane.

Preferentially, the compound of formula (11) is added to the reactor where step A3 takes place in the form of an aqueous solution or else in the form of a salt. Preferentially, the hydroxylamine is added in salt form.

Preferentially, the hydroxylamine in salt form is chosen from the group formed by hydroxylamine sulfate salts, hydroxylamine chloride salts and the mixtures of these salts. In the case of the use of hydroxylamine in salt form, a base will preferentially be able to be added to the reaction medium. As examples of a base, mention may be made of sodium acetate or triethylamine. The amount of base added will be within a range extending from 1 to 2 molar equivalents relative to the hydroxylamine generated, preferentially from 1 to 1.2 molar equivalents relative to the hydroxylamine generated. “Hydroxylamine generated” is understood to mean the cation (NH3⁺OH) of the hydroxylamine salt which is liberated when said salt is brought into contact with water. When a base is used, said base is mixed with the hydroxylamine salt and the mixture is then dissolved in water.

Preferentially, the organic solvent S3 is identical to the organic solvent S2. Thus, when the organic solvent S2 is a halogenated solvent, preferably a chlorinated solvent, more preferentially dichloromethane, then S3 is also a halogenated solvent, preferably a chlorinated solvent, more preferentially dichloromethane.

Preferentially, the reaction A3 is carried out in the presence of a cosolvent, said cosolvent being chosen from the group consisting of alcohols such as ethanol or isopropanol. More preferentially still, the organic solvent S3 is a halogenated solvent and the cosolvent is an alcohol, more preferentially the organic solvent S3 is a chlorinated solvent and the cosolvent is an alcohol. More preferentially still, the organic solvent S3 is dichloromethane and the cosolvent is an alcohol, preferably isopropanol or ethanol.

The synthesis process of the invention may comprise an optional step A4 of isolating, purifying and drying the compound of formula (I) in order to recover the compound of formula (I).

Preferentially, among the compounds of formula (I) which are synthesized according to the process of the invention described above, the compound of formula (Ia) is more particularly preferred

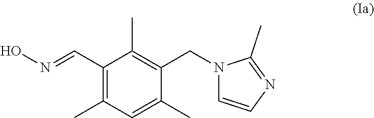 [SMILES: Cc1cc(C)c(Cn2ccnc2C)c(C)c1/C=N/O]

A second subject of the present invention relates to a process for synthesizing a compound of formula (X), said process comprising the following steps:
a step (i) of synthesizing the compound of formula (I) via the synthesis process as described above,
a step (ii) of converting the compound of formula (I) into a compound of formula (X) in the presence of at least one organic solvent S4 and at least one oxidant according to the following reaction scheme:

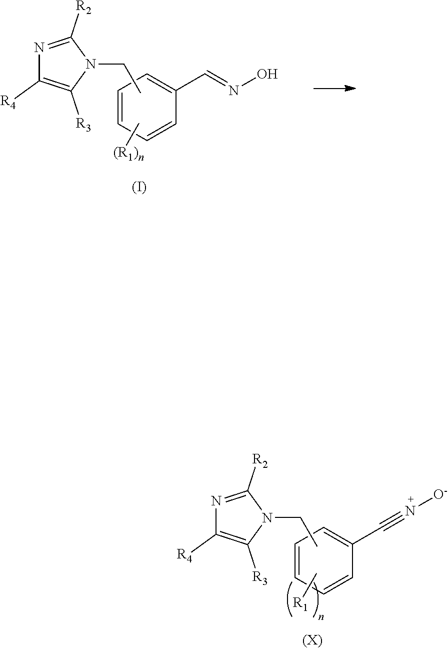

where:
R₁ represents, independently of one another, a C1-C6 alkyl, preferably a C1-C3 alkyl;
R₂ represents a hydrogen atom or a C1-C12 alkyl, preferably a C1-C6 alkyl, more preferentially a C1-C3 alkyl;
R₃ and R₄, independently of one another, represent a hydrogen atom, a C1-C12 alkyl, preferably a C1-C6 alkyl, more preferentially a C1-C3 alkyl, or form, together with the carbon atoms to which they are attached, a ring, preferably an aromatic ring, more preferentially phenyl; and
n is an integer equal to 0, 1, 2, 3 or 4; and
   a step (iii) of recovering the compound of formula (X).

Preferentially, in the compound of formula (I) and in the compound of formula (III), n is an integer equal to 3; R₁ represents, independently of one another, a C1-C6 alkyl, preferably a C1-C3 alkyl; R₂ represents a hydrogen atom or a C1-C6 alkyl, more preferentially a C1-C3 alkyl; and R₃ and R₄ represent a hydrogen atom or a C1-C6 alkyl, more preferentially a C1-C3 alkyl, or form, together with the carbon atoms to which they are attached, a phenyl. More preferentially still, n is an integer equal to 3; R₁ is a C1-C3 alkyl; R₂ represents a C1-C3 alkyl, preferably methyl, ethyl or propyl; and R₃ and R₄ represent a hydrogen atom or a C1-C3 alkyl (preferably methyl, ethyl or propyl) or form, together with the carbon atoms to which they are attached, a phenyl. Even more preferably, n is equal to 3; the R₁ groups are identical, located in the ortho and para position with respect to the oxime function, respectively the nitrile oxide function, and are a C1-C3 alkyl, preferably methyl, ethyl or propyl; R₂ represents a C1-C3 alkyl, preferably methyl, ethyl or propyl; and R₃ and R₄ are identical and are a hydrogen atom.

Step (i) as well as these preferred embodiments have been described above.

Preferentially, in step (ii), the oxidant is chosen from the group consisting of sodium hypochlorite, N-bromosuccinimide in the presence of a base, N-chlorosuccinimide in the presence of a base; preferentially the oxidant is sodium hypochlorite.

Preferentially, the organic solvent S4 is chosen from the group formed by alcohols such as ethanol or isopropanol, chlorinated solvents such as chloroform or dichloromethane, ethyl acetate.

Advantageously, the amount of oxidizing agent is from 1 to 5 molar equivalents, preferentially from 1 to 2 molar equivalents, relative to the molar amount of oxime of formula (I).

Preferentially, among the compounds of formula (X) which are synthesized according to the process of the invention described above, the compound of formula (Xa) is more particularly preferred

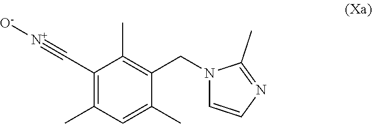 [SMILES: Cc1cc(C)c(Cn2ccnc2C)c(C)c1C#[N+][O-]]

The examples given below are presented by way of illustration and have no limiting effect on the invention.

### EXAMPLES

### Test 1:

### 1.1—Synthesis of the Compound of Formula (Ia) 2,4,6-trimethyl-3-((2-methyl-1H-imidazol-1-yl)methyl)benzaldehyde oxime According to the Process of the Invention (Process without Isolation of Synthesis Intermediates B and C))

The compound of formula (I) is synthesized according to the following reaction scheme:

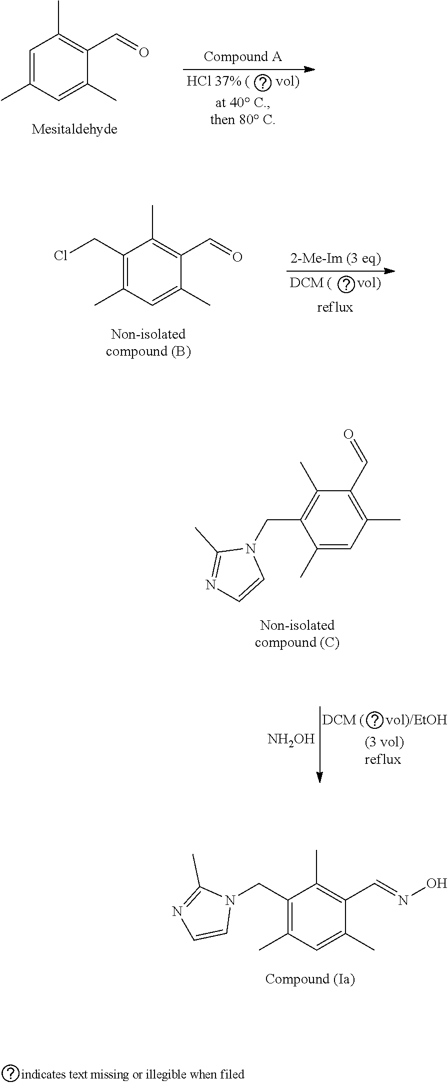

5.58 l of a hydrochloric acid solution (37% in solution in water) and 0.57 kg of paraformaldehyde (compound A, 18.82 mol, 2.5 eq. (eq. =molar equivalent)) are mixed in a 25 l reactor at 40° C. After stirring for 10 min at 40° C., the mixture becomes homogeneous and 1.12 kg of mesitaldehyde (7.53 mol) is added. The mixture is kept stirring and heated to 80° C. gradually, that is to say 2° C./min. After stirring for 4 hours at 80° C., the mixture is cooled to 40° C. and diluted with dichloromethane (1.12 l). The organic phase is separated and used in the following step without additional purification.

1.75 kg of a solution of 2-methyl-1H-imidazole ((2-Me-Im), 21.3 mol) in dichloromethane (5.92 l) is brought to reflux in order to achieve partial solubilization, then the previously prepared solution of 3-(chloromethyl)-2,4,6-trimethylbenzaldehyde (compound B) in dichloromethane (7.53 mol in 1.12 l of dichloromethane) is added to the 2-methyl-1H-imidazole solution. An orange mixture is obtained which is kept at reflux with stirring for 2 hours. The heating is stopped and the reaction mixture is washed 5 times with 5.25 l of water. The organic phase is separated and used in the following step without additional treatment (that is to say without purification and without isolation).

In a 50 l reactor, 4.38 l of ethanol and 1.81 l of dichloromethane are added to the previously obtained solution of 2,4,6-trimethyl-3-[(2-methyl-1H-imidazol-1-yl)methyl]benzaldehyde in dichloromethane; then 0.35 l of an aqueous solution of hydroxylamine at 50% by mass in water (6 mol) is added. The mixture is heated in order to be brought to reflux and is kept stirring for 7 hours. The suspension is subsequently cooled in order to reach the ambient temperature of 23° C., and then concentrated and filtered. The solid thus obtained is subsequently washed on the filter by the addition of 0.58 l of ethanol and then dried under vacuum for one day. After drying under vacuum, a white solid is recovered with an overall yield over the 3 steps of 65% (1.26 kg, 4.9 mol).

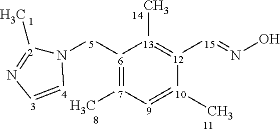

TABLE 1

| No. | δ ¹H (ppm) | δ ¹³C (ppm) |
|---|---|---|
| 1 | 2.31 | 12.7 |
| 2 | (—) | 143.4 |
| 3 | 6.58 | 125.8 |
| 4 | 6.22 | 116.9 |
| 5 | 4.97 | 43.2 |
| 6 | (—) | 129.3 |
| 7 | (—) | 136.2 |
| 8 | 2.23 | 20.2 |
| 9 | 6.97 | 130 |
| 10 | (—) | 137.3 |
| 11 | 2.15 | 19.1 |
| 12 | (—) | 129.1 |
| 13 | (—) | 136.1 |
| 14 | 2.11 | 15.9 |
| 15 | 8.25 | 147.4 |
| OH | 11.11 | (—) |

### 1.2—Synthesis of the Compound of formula (I) 2,4,6-trimethyl-3-((2-methyl-1H-imidazol-1-yl)methyl)benzaldehyde oxime According to a Process of the Prior Art Described in the Examples of WO2015059269

The 2,4,6-trimethyl-3-((2-methyl-1H-imidazol-1-yl)methyl)benzaldehyde oxime is synthesized according to a process of the prior art according to the reaction scheme below.

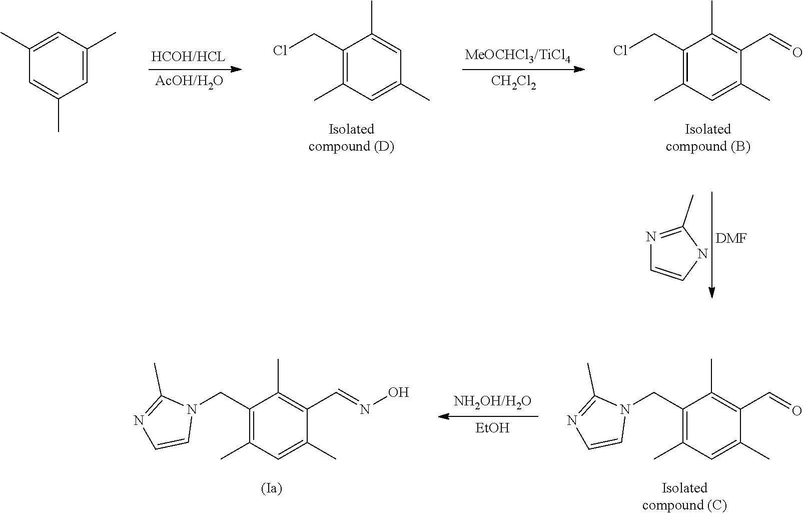

### 1.2.1—Synthesis of 2-(chloromethyl)-1,3,5-trimethylbenzene (Compound D)

This compound can be obtained according to a procedure described in the following paper: Zenkevich, I. G.; Makarov, A. A.; Russian Journal of General Chemistry; Vol. 77; No. 4; (2007); p. 611-619 (Zhurnal Obshchei Khimii; Vol. 77; No. 4; (2007); p. 653-662).

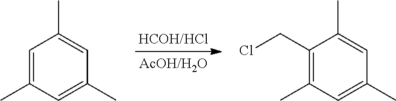

A mixture of mesitylene (100.0 g, 0.832 mol), paraformaldehyde (26.2 g, 0.874 mol) and hydrochloric acid (240 ml, 37%, 2.906 mol) in acetic acid (240 ml) is stirred and heated very slowly (1.5 hours) up to 37° C. After returning to ambient temperature, the mixture is diluted with water (1.0 l) with CH₂Cl₂ (200 ml) and the product is extracted with CH₂Cl₂ (4 times with 50 ml). The organic phases are combined, then washed with water (5 times with 100 ml) and evaporated down to 11-12 mbar (temperature of the bath=42° C.). A colourless oil (133.52 g, yield 95%) is obtained. After 15-18 hours at +4° C., the oil crystallized. The crystals are filtered off, washed with petroleum ether cooled to −18° C. (40 ml), then dried under atmospheric pressure at ambient temperature for 3 to 5 hours. A white solid (95.9 g, yield 68%) with a melting point of 39° C. is obtained. The molar purity is greater than 96% (¹H NMR).

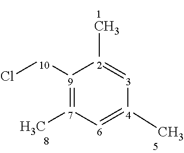

TABLE 2

| No. | δ ¹H (ppm) | δ ¹³C (ppm) |
|---|---|---|
| 1 | 2.27 | 18.4 |
| 2 | (—) | 136.9 |
| 3 | 6.81 | 128.5 |
| 4 | (—) | 137.4 |
| 5 | 2.15 | 20.3 |
| 6 | 6.81 | 128.5 |
| 7 | (—) | 136.9 |
| 8 | 2.27 | 18.4 |
| 9 | (—) | 130.5 |
| 10 | 4.69 | 41.3 |

### 1.2.2—Synthesis of 3-(chloromethyl)-2,4,6-trimethylbenzaldehyde (Compound B)

This compound can be obtained according to a procedure described in the following paper: Yakubov, A. P.; Tsyganov, D. V.; Belen'kii, L. I.; Krayushkin, M. M.; Bulletin of the Academy of Sciences of the USSR, Division of Chemical Science (English Translation); Vol. 40; No. 7.2; (1991); p. 1427-1432 (Izvestiya Akademii Nauk SSSR, Seriya Khimicheskaya; No. 7; (1991); p. 1609-1615).

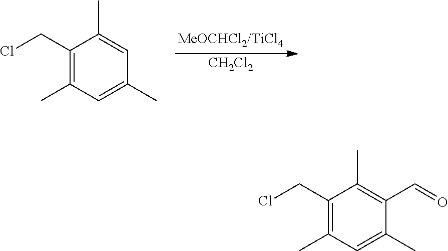

A solution of 2-(chloromethyl)-1,3,5-trimethylbenzene (20.0 g, 0.118 mol) and dichloromethyl methyl ether (27.26 g, 0.237 mol) in dichloromethane (200 ml) is added under argon over 10-12 minutes to a solution of TiCl₄ (90.0 g, 0.474 mol) in dichloromethane (200 ml) at 17° C. After stirring at 17-20° C. for 15-20 minutes, water (1000 ml) and ice (500 g) are added to the reaction medium. After stirring for 10-15 minutes, the organic phase is separated. The aqueous phase is extracted with CH₂Cl₂ (3 times with 75 ml). The combined organic phases are washed with water (4 times with 100 ml) and evaporated under reduced pressure to result in a solid (temperature of the bath=28° C.). The target product (22.74 g) is obtained with a yield of 97%. Its melting point is 58° C. The molar purity, estimated by ¹H NMR, is 95 mol %.

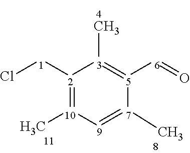

TABLE 3

| No. | δ ¹H (ppm) | δ ¹³C (ppm) |
|---|---|---|
| 1 | 4.77 | 40.6 |
| 2 | (—) | 132.9 |
| 3 | (—) | 139.5 |
| 4 | 2.51 | 14.4 |
| 5 | (—) | 131.4 |
| 6 | 10.43 | 194.2 |
| 7 | (—) | 140.1 |
| 8 | 2.41 | 19.3 |
| 9 | 6.99 | 131.2 |
| 10 | (—) | 142.4 |
| 11 | 2.34 | 19.8 |

### 1.2.3—Synthesis of 2,4,6-trimethyl-3-((2-methyl-1H-imidazol-1-yl)methyl)benzaldehyde (Compound C)

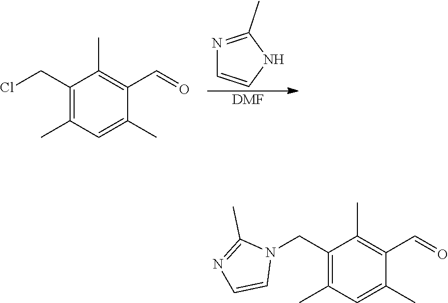

A mixture of 3-(chloromethyl)-2,4,6-trimethylbenzaldehyde (10.0 g, 0.051 mol) and imidazole (10.44 g, 0.127 mol) in DMF (10 ml) is stirred at 80° C. for one hour.

After returning to 40-50° C., the mixture is diluted with water (200 ml) and stirred for 10 minutes. The precipitate obtained is filtered off, washed on the filter with water (4 times with 25 ml), then dried at ambient temperature. A white solid (7.92 g, yield 64%) with a melting point of 161° C. is obtained. The molar purity is 91% (¹H NMR).

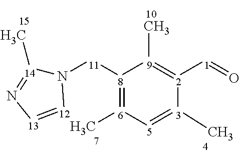

TABLE 4

| No. | δ ¹H (ppm) | δ ¹³C (ppm) |
|---|---|---|
| 1 | 10.45 | 194.2 |
| 2 | (—) | 131.5 |
| 3 | (—) | 139.5 |
| 4 | 2.44 | 19.6 |
| 5 | 7.04 | 131.2 |
| 6 | (—) | 142.5 |
| 7 | 2.19 | 19.5 |
| 8 | (—) | 131 |
| 9 | (—) | 139.5 |
| 10 | 2.34 | 14.6 |
| 11 | 5.02 | 42.5 |
| 12 | 6.24 | 116.9 |
| 13 | 6.59 | 125.9 |
| 14 | (—) | 143.5 |
| 15 | 2.32 | 12.7 |

### 1.2.4—Synthesis of 2,4,6-trimethyl-3-((2-methyl-1H-imidazol-1-yl)methyl)benzaldehyde oxime (Compound Ia)

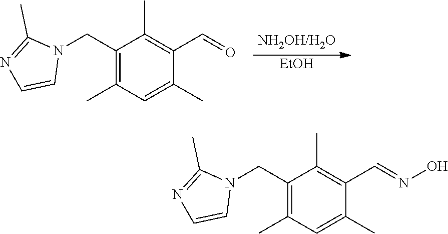

An aqueous hydroxylamine solution (809 g, 0.134 mol, 50% in water, Aldrich) in EtOH (10 ml) is added to a solution of 2,4,6-trimethyl-3-((2-methyl-1H-imidazol-1-yl)methyl)benzaldehyde (20.3 g, 0.084 mol) in EtOH (110 ml) at 40° C. The reaction medium is stirred at a temperature of 50 to 55° C. for 2.5 hours. After returning to 23° C., the precipitate obtained is filtered off, washed twice on the filter with an EtOH/H₂O (10 ml/15 ml) mixture and dried under atmospheric pressure at ambient temperature for 15 to 20 hours. A white solid (19.57 g, yield 91%) with a melting point of 247° C. is obtained. The molar purity is greater than 87% (¹H NMR).

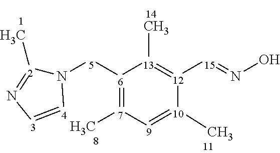

TABLE 5

| No. | δ ¹H (ppm) | δ ¹³C (ppm) |
|---|---|---|
| 1 | 2.31 | 12.7 |
| 2 | (—) | 143.4 |
| 3 | 6.58 | 125.8 |
| 4 | 6.22 | 116.9 |
| 5 | 4.97 | 43.2 |
| 6 | (—) | 129.3 |
| 7 | (—) | 136.2 |
| 8 | 2.23 | 20.2 |
| 9 | 6.97 | 130 |
| 10 | (—) | 137.3 |
| 11 | 2.15 | 19.1 |
| 12 | (—) | 129.1 |
| 13 | (—) | 136.1 |
| 14 | 2.11 | 15.9 |
| 15 | 8.25 | 147.4 |
| OH | 11.11 | (—) |

### 1.3—Comparison of the Process of the Invention and the Process of the Prior Art

TABLE 6

| | | Synthesis of | Synthesis of |
|---|---|---|---|
| | | compound (Ia) | compound (Ia) |
| | | according to the | according to the |
| | | process of the | process of the |
| | | prior art | invention |
| | | (process 1.2) | (process 1.1) |
| Number of intermediates isolated | | 3 | 0 |
| Overall yield | | 38% | 65% |
| Number of solvents used | | 4 | 2 |
| Litres of solvent | dichloromethane | 71.4 | 7 |
| used/kg of final | alcohol (ethanol, | 7.2 | 3.9 |
| product | isopropanol) | | |
| | petroleum ether | 4.4 | 0 |
| | DMF | 1.3 | 0 |

| | | |
|---|---|---|
| Litres of aqueous derivatives (water, HCl) | 190.2 | 25.5 |
| used/kg of final product | | |

Completely unexpectedly, the process according to the invention for synthesizing the compound of formula (Ia) enables a reduction in the number of intermediates isolated while improving the yield of the process. It also enables a decrease in the number of solvents used, therefore a simplification of handling by an operator and an improvement in the overall cycle time by eliminating the times associated with the steps of purifying and drying the intermediates. Finally, it enables a decrease in the effluents and therefore also in the treatments thereof.

### Test 2:

2.1—Process for synthesizing the compound of formula (Xa) from the compound of formula (Ia) synthesized according to the process of the invention (in accordance with the invention) The compound of formula (Xa) is obtained from the compound of formula (Ia) obtained by the process without isolation of the intermediates of the invention and according to the following reaction scheme:

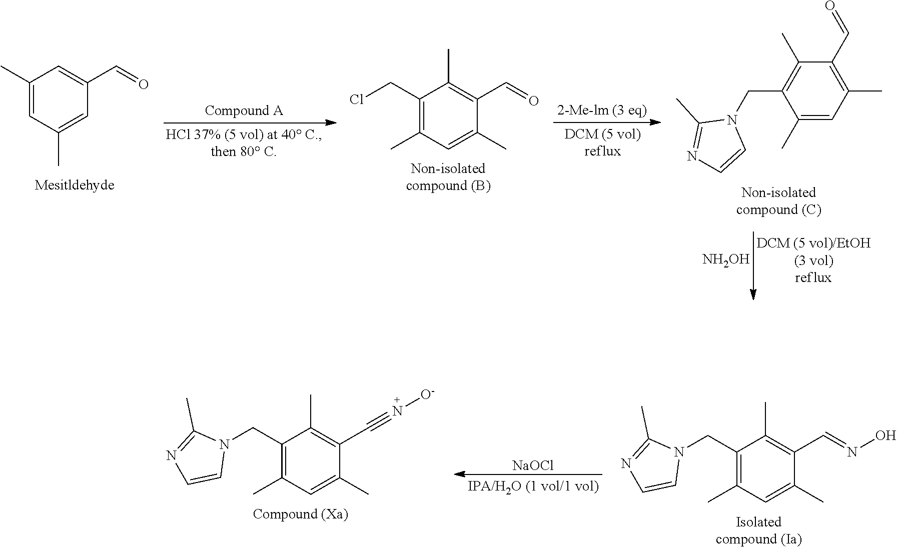

1.26 kg of 2,4,6-trimethyl-3-((2-methyl-1H-imidazol-1-yl)methyl)benzaldehyde oxime (4.9 mol), compound (Ia) obtained according to the process without isolation of the intermediates of the invention as described in paragraph 1.1 above, is suspended in isopropanol/water (1.3 l/1.3 l). The mixture is cooled to 0° C. and 6.44 l of a 14% sodium hypochlorite solution in water (14.7 mol) is added over a period of 30 min. At the end of the addition, the mixture is kept stirring at 2-3° C. for 2 hours.

The product is subsequently filtered off and the beige powder is resuspended in water (4.73 l) and stirred for 1 h then filtered. The operation is repeated 4 times. After the last filtration, the powder is ground and dried under vacuum (40 mbar) for 8 h at 50° C. and then overnight at 40° C. under vacuum (50 mbar). The 2,4,6-trimethyl-3-((2-methyl-1H-imidazol-1-yl)methyl)benzonitrile N-oxide is obtained with a yield of 80% (1 kg, 3.92 mol), in the form of a slightly yellow solid.

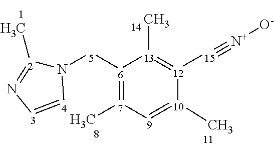

TABLE 7

| No. | δ ¹H (ppm) | δ ¹³C (ppm) |
|---|---|---|
| 1 | 2.3 | 12.6 |
| 2 | (—) | 143.6 |
| 3 | 6.59 | 126.1 |
| 4 | 6.27 | 117.1 |
| 5 | 4.99 | 43 |
| 6 | (—) | 130.6 |
| 7 | (—) | 140.7 |
| 8 | 2.16 | 19.2 |
| 9 | 7.12 | 129.9 |
| 10 | (—) | 141 |
| 11 | 2.34 | 20 |
| 12 | (—) | 112.1 |
| 13 | (—) | 140.8 |
| 14 | 2.28 | 17.7 |
| 15 | (—) | NI |

### 2.2—Synthesis of the Compound of Formula (Xa) According to a Process of the Prior Art (not in Accordance with the Invention)

The 2,4,6-trimethyl-3-2 ((2-methyl-1H-imidazol-1-yl)benzonitrile N-oxide (compound Xa) is prepared according to the following reaction scheme:

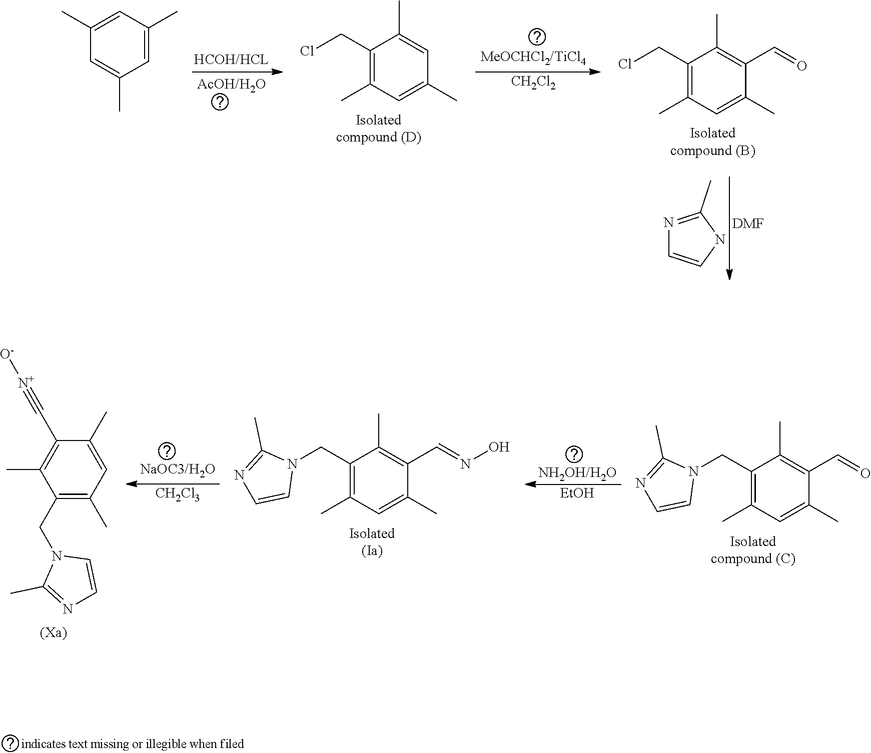

### 2.2.1 Synthesis of 2-(chloromethyl)-1,3,5-trimethylbenzene (Compound (D))

This compound can be obtained according to a procedure described in the following paper: Zenkevich, 1. G.; Makarov, A. A.; Russian Journal of General Chemistry; Vol. 77; No. 4; (2007); p. 611-619 (Zhurnal Obshchei Khimii; Vol. 77; No. 4; (2007); p. 653-662).

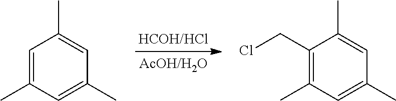

A mixture of mesitylene (100.0 g, 0.832 mol), paraformaldehyde (26.2 g, 0.874 mol) and hydrochloric acid (240 ml, 37%, 2.906 mol) in acetic acid (240 ml) is stirred and heated very slowly (1.5 hours) up to 37° C. After returning to ambient temperature, the mixture is diluted with water (1.01l) with CH2Cl2 (200 ml) and the product is extracted with CH2Cl2 (4 times with 50 ml). The organic phases are combined, then washed with water (5 times with 100 ml) and evaporated down to 11-12 mbar (temperature of the bath=42° C.). A colourless oil (133.52 g, yield 95%) is obtained. After 15-18 hours at +4° C., the oil crystallized. The crystals are filtered off, washed with petroleum ether cooled to −18° C. (40 ml), then dried under atmospheric pressure at ambient temperature for 3 to 5 hours. A white solid (95.9 g, yield 68%) with a melting point of 39° C. is obtained. The molar purity is greater than 96% (¹H NMR).

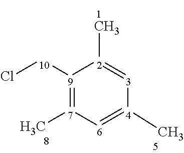

TABLE 8

| No. | δ ¹H (ppm) | δ ¹³C (ppm) |
|---|---|---|
| 1 | 2.27 | 18.4 |
| 2 | (—) | 136.9 |
| 3 | 6.81 | 128.5 |
| 4 | (—) | 137.4 |
| 5 | 2.15 | 20.3 |
| 6 | 6.81 | 128.5 |
| 7 | (—) | 136.9 |
| 8 | 2.27 | 18.4 |
| 9 | (—) | 130.5 |
| 10 | 4.69 | 41.3 |

2.2.2—Synthesis of 3-(chloromethyl)-2,4,6-trimethylbenzaldehyde (Compound (B)) This compound can be obtained according to a procedure described in the following paper: Yakubov, A. P.; Tsyganov, D. V.; Belen'kii, L. I.; Krayushkin, M. M.; Bulletin of the Academy of Sciences of the USSR, Division of Chemical Science (English Translation); Vol. 40; No. 7.2; (1991); p. 1427-1432 (Izvestiya Akademii Nauk SSSR, Seriya Khimicheskaya; No. 7; (1991); p. 1609-1615)].

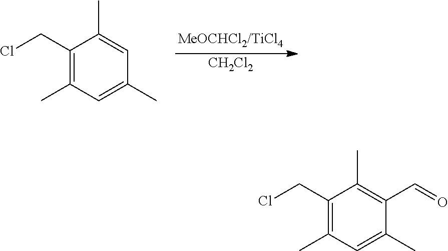

A solution of 2-(chloromethyl)-1,3,5-trimethylbenzene (20.0 g, 0.118 mol) and dichloromethyl methyl ether (27.26 g, 0.237 mol) in dichloromethane (200 ml) is added under argon over 10-12 minutes to a solution of TiCl₄ (90.0 g, 0.474 mol) in dichloromethane (200 ml) at 17° C. After stirring at 17-20° C. for 15-20 minutes, water (1000 ml) and ice (500 g) are added to the reaction medium. After stirring for 10-15 minutes, the organic phase is separated. The aqueous phase is extracted with CH₂Cl₂ (3 times with 75 ml). The combined organic phases are washed with water (4 times with 100 ml) and evaporated under reduced pressure to result in a solid (temperature of the bath=28° C.). The target product (22.74 g) is obtained with a yield of 97%. Its melting point is 58° C. The molar purity, estimated by ¹H NMR, is 95 mol %.

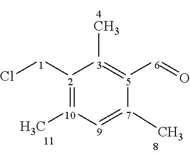

TABLE 9

| No. | δ ¹H (ppm) | δ ¹³C (ppm) |
|---|---|---|
| 1 | 4.77 | 40.6 |
| 2 | (—) | 132.9 |
| 3 | (—) | 139.5 |
| 4 | 2.51 | 14.4 |
| 5 | (—) | 131.4 |
| 6 | 10.43 | 194.2 |
| 7 | (—) | 140.1 |
| 8 | 2.41 | 19.3 |
| 9 | 6.99 | 131.2 |
| 10 | (—) | 142.4 |
| 11 | 2.34 | 19.8 |

### 2.2.3—Synthesis of 2,4,6-trimethyl-3-((2-methyl-1H-imidazol-1-yl)methyl)benzaldehyde (Compound (C))

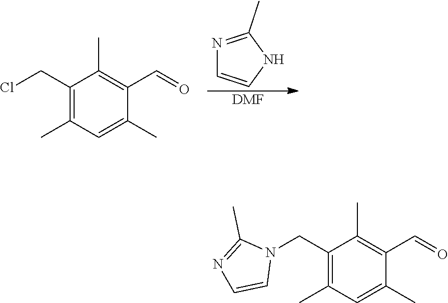

A mixture of 3-(chloromethyl)-2,4,6-trimethylbenzaldehyde (10.0 g, 0.051 mol) and imidazole (10.44 g, 0.127 mol) in DMF (10 ml) is stirred at 80° C. for one hour.

After returning to 40-50° C., the mixture is diluted with water (200 ml) and stirred for 10 minutes. The precipitate obtained is filtered off, washed on the filter with water (4 times with 25 ml), then dried at ambient temperature. A white solid (7.92 g, yield 64%) with a melting point of 161° C. is obtained. The molar purity is 91% (¹H NMR).

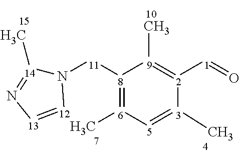

TABLE 10

| No. | δ ¹H (ppm) | δ ¹³C (ppm) |
|---|---|---|
| 1 | 10.45 | 194.2 |
| 2 | (—) | 131.5 |
| 3 | (—) | 139.5 |
| 4 | 2.44 | 19.6 |
| 5 | 7.04 | 131.2 |
| 6 | (—) | 142.5 |
| 7 | 2.19 | 19.5 |
| 8 | (—) | 131 |
| 9 | (—) | 139.5 |
| 10 | 2.34 | 14.6 |
| 11 | 5.02 | 42.5 |
| 12 | 6.24 | 116.9 |
| 13 | 6.59 | 125.9 |
| 14 | (—) | 143.5 |
| 15 | 2.32 | 12.7 |

### 2.2.4—Synthesis of 2,4,6-trimethyl-3-((2-methyl-1H-imidazol-1-yl)methyl)benzaldehyde oxime (Compound (Ia))

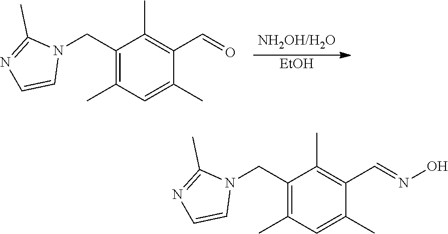

An aqueous hydroxylamine solution (809 g, 0.134 mol, 50% in water, Aldrich) in EtOH (10 ml) is added to a solution of 2,4,6-trimethyl-3-((2-methyl-1H-imidazol-1-yl)methyl)benzaldehyde (20.3 g, 0.084 mol) in EtOH (110 ml) at 40° C. The reaction medium is stirred at a temperature of 50 to 55° C. for 2.5 hours. After returning to 23° C., the precipitate obtained is filtered off, washed twice on the filter with an EtOH/H₂O (10 ml/15 ml) mixture and dried under atmospheric pressure at ambient temperature for 15 to 20 hours. A white solid (19.57 g, yield 91%) with a melting point of 247° C. is obtained. The molar purity is greater than 87% (¹H NMR).

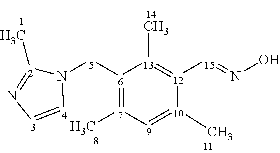

TABLE 11

| No. | δ ¹H (ppm) | δ ¹³C (ppm) |
|---|---|---|
| 1 | 2.31 | 12.7 |
| 2 | (—) | 143.4 |
| 3 | 6.58 | 125.8 |
| 4 | 6.22 | 116.9 |
| 5 | 4.97 | 43.2 |
| 6 | (—) | 129.3 |
| 7 | (—) | 136.2 |
| 8 | 2.23 | 20.2 |
| 9 | 6.97 | 130 |
| 10 | (—) | 137.3 |
| 11 | 2.15 | 19.1 |
| 12 | (—) | 129.1 |
| 13 | (—) | 136.1 |
| 14 | 2.11 | 15.9 |
| 15 | 8.25 | 147.4 |
| OH | 11.11 | (—) |

### 2.2.5—Synthesis of 2,4,6-trimethyl-3-((2-methyl-1H-imidazol-1-yl)methyl)benzonitrile oxide (Compound (Xa))

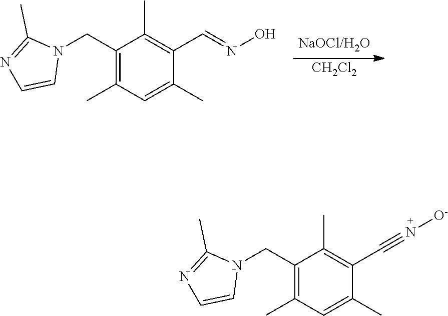

An aqueous solution of NaOCl (4% of active chlorine, Aldrich, 49 ml) is added dropwise over 5 minutes to a mixture of 2,4,6-trimethyl-3-((2-methyl-1H-imidazol-1-yl)methyl)benzaldehyde oxime (8.80 g, 0.034 mol) in CH₂Cl₂ (280 ml) at 6° C. The temperature of the reaction medium is maintained between 6° C. and 8° C. The reaction medium is subsequently stirred at 8° C. to 21° C. for 2 hours. The organic phase is separated. The organic phase is washed with water (3 times with 50 ml). After concentrating under reduced pressure (temperature of the bath=22-23° C., 220 mbar), petroleum ether (10 ml) is added, the solvent is evaporated down to 8-10 ml and the solution is maintained at −18° C. for 10-15 hours, so as to obtain a precipitate. The precipitate is filtered off, washed on the filter with the CH₂Cl2/petroleum ether (2 ml/6 ml) mixture and then with petroleum ether (2 times 10 ml), and finally dried under atmospheric pressure at ambient temperature for 10-15 hours. A white solid (5.31 g, yield 61%) with a melting point of 139° C. is obtained.

The molar purity is greater than 95 mol % (¹H NMR).

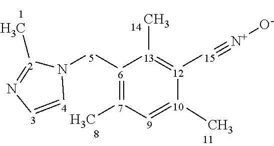

TABLE 12

| No. | δ ¹H (ppm) | δ ¹³C (ppm) |
|---|---|---|
| 1 | 2.3 | 12.6 |
| 2 | (—) | 143.6 |
| 3 | 6.59 | 126.1 |
| 4 | 6.72 | 117.1 |
| 5 | 4.99 | 43 |
| 6 | (—) | 130.6 |
| 7 | (—) | 140.7 |
| 8 | 2.16 | 19.2 |
| 9 | 7.12 | 129.9 |
| 10 | (—) | 141 |
| 11 | 2.34 | 20 |
| 12 | (—) | 112.1 |
| 13 | (—) | NI |
| 14 | (—) | 140.8 |
| 15 | 2.8 | 17.7 |

2.3—Comparison of the process for synthesizing the nitrile oxide (compound Xa) obtained by conversion of compound (Ia) obtained according to the process of the invention (paragraph 2.1) with respect to the process for synthesizing the nitrile oxide (compound Xa) according to the synthesis process of the prior art (paragraph 2.2):

TABLE 13

| | | | Process for |
|---|---|---|---|
| | | Process for | synthesizing |
| | | synthesizing | compound (Xa) |
| | | compound (Xa) of | according to the |
| | | the prior art | invention |
| | | (process 2.2) | (process 2.1) |
| Number of intermediates isolated | | 4 | 1 |
| overall yield | | 23% | 52% |
| number of solvents used | | 4 | 2 |
| Litres of solvent | dichloromethane | 117.9 | 8.9 |
| used/kg of final | Alcohol (ethanol, isopropanol) | 12 | 6.2 |
| product | petroleum ether | 7.3 | 0 |
| | DMF | 2.2 | 0 |

| | | |
|---|---|---|
| Litres of aqueous derivatives (water, HCl) used/kg of | 311 | 63.5 |
| final product | | |

Completely unexpectedly, the process for synthesizing the compound of formula (Xa) from the compound of formula (Ia) obtained according to the process of the invention enables a reduction in synthesis steps and in the number of intermediates isolated while improving the yield. It also enables a decrease in the number of solvents used, therefore a simplification of handling by an operator. Finally, it enables a decrease in the effluents and therefore also in the treatments thereof.

1.-15. (canceled)

16. A process for synthesizing a compound of formula (I) 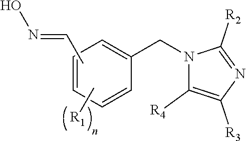
in which
R₁ represents, independently of one another, a C1-C6 alkyl;
R₂ represents a hydrogen atom or a C1-C12 alkyl;
R₃ and R₄, independently of one another, represent a hydrogen atom, a C1-C12 alkyl, or form, together with the carbon atoms to which they are attached, a ring; and
n is an integer equal to 0, 1, 2, 3 or 4,
the process comprising the following successive steps:
a step A1 of obtaining an intermediate compound of formula (V) via a halomethylation reaction of an aromatic aldehyde of formula (VI) in a presence of a halomethylation agent 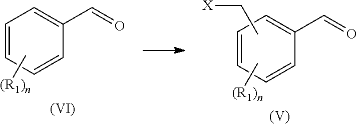
with R₁ and n as defined above; and X being a halogen atom;
a step A2 of obtaining an intermediate compound of formula (III) via nucleophilic substitution of the intermediate compound of formula (V) with an imidazole compound of formula (IV) 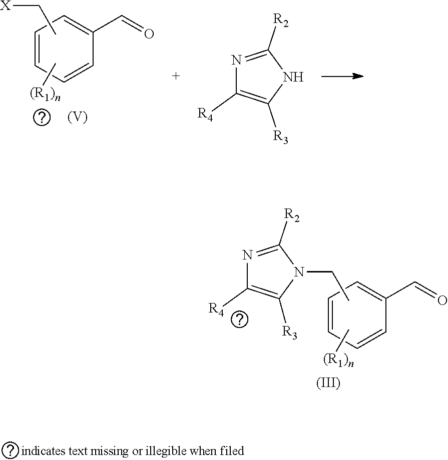
with n, R₁, R₂, R₃ and R₄ as defined above; and
a step A3 of obtaining the compound of formula (I) via a condensation reaction of the intermediate compound of formula (III) with hydroxylamine (II) 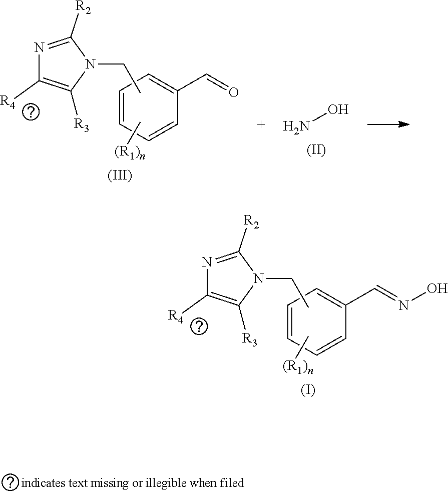
with R₁, R₂, R₃, R₄ and n as defined above,
wherein steps A1, A2, A3 are carried out without isolation of at least one intermediate compound selected from the group consisting of the intermediate compound of formula (III) and the intermediate compound of formula (V).

17. The process according to claim 16, wherein the halomethylation agent is preheated to a temperature T1 within a range extending from 23° C. to 50° C. before bringing the halomethylation agent into contact with the compound of formula (VI).

18. The process according to claim 17, wherein the compound of formula (VI) is added all at once after the preheating of the halomethylation agent.

19. The process according to claim 16, wherein the halomethylation agent is a mixture of a formaldehyde donor and hydrochloric acid.

20. The process according to claim 19, wherein an amount of formaldehyde donor is within a range extending from 1 molar equivalent to 7 molar equivalents relative to an amount of the compound of formula (VI).

21. The process according to claim 16, wherein step A1 is carried out at a temperature T2 within a range extending from 60° C. to 100° C.

22. The process according to claim 16, wherein an organic solvent S1 is added at an end of step A1 and an organic phase is recovered, the organic solvent S1 being a halogenated solvent.

23. The process according to claim 16, wherein step A2 is carried out in a presence of an organic solvent S2 selected from the group consisting of halogenated solvents, dimethyl sulfoxide, acetone, alcohols, ethers, and ethyl acetate.

24. The process according to claim 23, wherein an organic solvent S1 is added at an end of step A1 and an organic phase is recovered, the organic solvent S1 being a halogenated solvent, and
wherein the organic solvent S2 is identical to the organic solvent S1.

25. The process according to claim 16, wherein an amount of the compound of formula (IV) is within a range extending from 1.1 molar equivalents to 6 molar equivalents relative to the compound of formula (V).

26. The process according to claim 23, wherein step A2 is carried out at a temperature which is less than or equal to a reflux temperature of the organic solvent S2.

27. The process according to claim 23, wherein an organic solvent S1 is added at an end of step A1 and an organic phase is recovered, the organic solvent S1 being a halogenated solvent, and
wherein an amount of organic solvent S2 is at least 0.5 volume relative to 1 volume of organic solvent S1.

28. The process according to claim 16, wherein step A3 is carried out in an organic solvent S3 selected from the group consisting of halogenated solvents, acetone, alcohols, ethers, and ethyl acetate.

29. The process according to claim 28, wherein step A2 is carried out in a presence of an organic solvent S2 selected from the group consisting of halogenated solvents, dimethyl sulfoxide, acetone, alcohols, ethers, and ethyl acetate, and
wherein the organic solvent S3 is identical to the organic solvent S2.

30. A process for synthesizing a compound of formula (X), the process comprising the following steps:
a step (i) of synthesizing the compound of formula (I) via the process according to claim 16;
a step (ii) of converting the compound of formula (I) into a compound of formula (X) in a presence of at least one organic solvent S4 and at least one oxidant 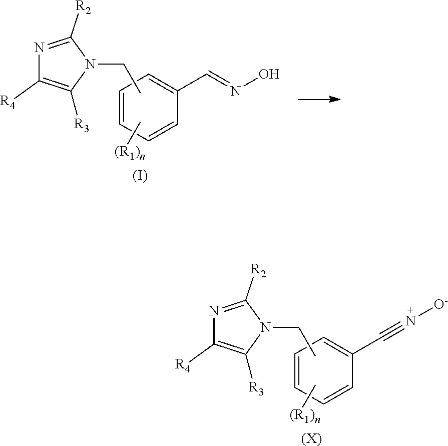
where
R₁ represents, independently of one another, a C1-C6 alkyl,
R₂ represents a hydrogen atom or a C1-C12 alkyl,
R₃ and R₄, independently of one another, represent a hydrogen atom, a C1-C12 alkyl, or form, together with the carbon atoms to which they are attached, a ring, and
n is an integer equal to 0, 1, 2, 3 or 4; and
a step (iii) of recovering the compound of formula (X).
